(19) United States
(12) Patent Application Publication
(10) Pub. No.: US 2020/0152080 A1
(43) Pub. Date: May 14, 2020

(54) SYSTEMS AND METHODS FOR KETOSIS BASED DIET MANAGEMENT

(71) Applicant: Keyto, Inc., San Francisco, CA (US)

(72) Inventors: Ray WU, San Francisco, CA (US); Liane NAKAMURA, Culver City, CA (US); Ethan J. WEISS, San Francisco, CA (US)

(73) Assignee: Keyto, Inc., San Francisco, CA (US)

(21) Appl. No.: 16/680,206

(22) Filed: Nov. 11, 2019

Related U.S. Application Data
(60) Provisional application No. 62/759,918, filed on Nov. 12, 2018.

(51) Int. Cl. G09B 19/00; A61B 5/08; A61B 5/00; A61B 5/097; G06Q 30/06; G16H 20/60

(52) U.S. Cl. CPC G09B 19/0092; A61B 5/082; A61B 5/0022; A61B 2560/0252; G06Q 30/0631; G16H 20/60; A61B 2560/0228; A61B 5/097

(57) ABSTRACT

A system for determining a level of ketosis of a user includes an acetone detector that includes an inlet for receiving a breath of a user, a sensor for sensing an amount of acetone in the breath of the user, a memory storing calibration values for calibrating sensor data, wherein the one or more calibration values are specific to the acetone detector; and an electronic device comprising programs configured for receiving from the acetone detector measurements of an amount of acetone in breath of a user, receiving from the acetone detector the calibration values, determining a ketosis score based on at least one of the measurements, the calibration values, and predetermined thresholds that are associated with levels of ketosis, wherein the ketosis score is an estimate of a level of ketosis of the user, and displaying the ketosis score to the user.

### CROSS-REFERENCE TO RELATED APPLICATIONS

This application claims the benefit of U.S. Provisional Application No. 62/759,918, filed Nov. 12, 2018, the entire contents of which is incorporated herein by reference.

### FIELD OF THE INVENTION

The present disclosure generally relates to systems and methods for monitoring breath acetone levels and, more specifically, to systems and methods for guiding a user's diet based on breath acetone levels.

### BACKGROUND OF THE INVENTION

Obesity is an ever-increasing public health concern. Studies have shown obesity to be a leading cause of diabetes, hypercholesterolemia and other disorders that lead to kidney and liver failure, and heart disease. Numerous diets employing special nutrient preparations or food choice prescriptions are available and widely used, e.g. Weight Watcher's (WW), NutriSystem, New Beverly Hills, Jenny Craig, the Pritikin Principle diet, etc. Each of these weight loss programs aims to force the body to burn fat stores. People who are on a diet (or diet plus exercise) regimen, generally monitor their own fat loss progress by total weight loss, as measured on weighing scales. However, loss of body fat often cannot be reliably measured by simple weighing because body fluid balance in particular, as well as other factors, may play a greater role in total body weight fluctuations than fat loss.

Dieting programs are often difficult for people to consistently follow. People may have difficulty staying motivated over the long period of time needed to achieve a weight loss goal. Daily fluctuations in weight due to factors other than weight loss or gain, such as water loss, may lead to reduced motivation and cessation of dieting.

The ketogenic diet has been shown to be effective in achieving weight loss. The ketogenic diet is a low-carbohydrate, high-fat diet that involves reducing carbohydrate intake and replacing with fat. This reduction in carbohydrates puts the body into a metabolic state called ketosis. During ketosis, cells use ketones generated from the metabolism of fat for energy rather than the blood sugar that comes from carbohydrates. Once ketosis is reached, most cells will use ketone bodies to generate energy until blood sugar from carbohydrates is available. Ketogenic diets seek to maintain ketosis for extended periods of time to maximum stored fat loss.

Acetone production is a by-product of the fat metabolism process and appears in exhaled breath. Acetone has been measured in exhaled breath to monitor ketosis in subjects. Breath acetone concentration has been shown to correlate with the rate of fat loss in healthy individuals. Accordingly, measurements of breath acetone levels can be used to estimate whether the body is using fat as a primary source of energy.

### SUMMARY OF THE INVENTION

According to some embodiments, systems and methods include measuring the amount of acetone in a user's breath using a breath analyzer and determining a level of ketosis of the user for guiding the user's diet. In some embodiments, a software application running on a user's smartphone wirelessly connects with the breath analyzer to receive acetone measurements taken when the user blows into the breath analyzer. The application may analyze the measurements to determine a ketosis score, which can be an indicator of the level of ketosis of the user that is qualitatively useful to the user. This ketosis score can be displayed to the user so that the user can gauge the progress of the user's diet. The ketosis score can be used to generate dietary and lifestyle recommendations to the user. Thus, systems and methods described herein can help a user to maintain a fat reducing diet through feedback, motivation, and guidance.

According to some embodiments, a system for determining a level of ketosis of a user includes an acetone detector comprising an inlet for receiving a breath of a user, a sensor for sensing an amount of acetone in the breath of the user, a memory storing one or more calibration values for calibrating sensor data, wherein the one or more calibration values are specific to the acetone detector; and an electronic device comprising a display, one or more processors, memory, and one or more programs configured for execution by the one or more processors, the one or more programs including instructions for: receiving from the acetone detector one or more measurements of an amount of acetone in breath of a user, receiving from the acetone detector the one or more calibration values, determining a ketosis score based on at least one of the one or more measurements, the one or more calibration values, and one or more predetermined thresholds that are associated with levels of ketosis, wherein the ketosis score is an estimate of a level of ketosis of the user, and displaying the ketosis score to the user.

In any of these embodiments, the acetone detector and the electronic device may communicate wirelessly.

In any of these embodiments, the ketosis score may be determined by calibrating the one or more measurements using the one or more calibration values to generate one or more calibrated measurements, transforming the one or more calibrated measurements using a transform to generate one or more transformed measurements, and comparing the one or more transformed measurements the one or more thresholds.

In any of these embodiments, the ketosis score may be determined based on one or more thresholds associated with levels of ketosis.

In any of these embodiments, the one or more threshold may include no more than 10 thresholds.

In any of these embodiments, the one or more programs may include instructions for transmitting the ketosis score to a remote server system that comprises dietary and/or lifestyle information and receiving a dietary and/or lifestyle recommendation generated for the user based on the ketosis score.

In any of these embodiments, the dietary recommendation may be generated based on one or more attributes of the user.

In any of these embodiments, the one or more attributes of the user may include one or more of a weight loss goal, age, gender, dietary preference, and weight.

In any of these embodiments, the dietary recommendation may be generated based on a previous ketosis score for the user.

According to some embodiments, a method for determining a level of ketosis of a user on an electronic device includes receiving from an acetone detector one or more measurements of an amount of acetone in breath of a user; receiving from the acetone detector one or more calibration values for calibrating acetone detector data, wherein the one or more calibration values are specific to the acetone detector; determining a ketosis score based on at least one of the one or more measurements, the one or more calibration values, and one or more predetermined thresholds that are associated with levels of ketosis, wherein the ketosis score is an estimate of a level of ketosis of the user; and displaying the ketosis score to the user.

In any of these embodiments, the electronic device may receive data wirelessly from the acetone detector.

In any of these embodiments, the ketosis score may be determined by calibrating the one or more measurements using the one or more calibration values to generate one or more calibrated measurements, transforming the one or more calibrated measurements using a transform to generate one or more transformed measurements, and comparing the one or more transformed measurements to the one or more thresholds.

In any of these embodiments, the ketosis score may be determined based on one or more thresholds associated with levels of ketosis.

In any of these embodiments, the one or more threshold may include no more than 10 thresholds.

In any of these embodiments, the method may include transmitting the ketosis score to a remote server system that comprises dietary and/or lifestyle information and receiving a dietary and/or lifestyle recommendation generated for the user based on the ketosis score.

In any of these embodiments, the dietary recommendation may be generated based on one or more attributes of the user.

In any of these embodiments, the one or more attributes of the user may include one or more of a weight loss goal, age, gender, dietary preference and weight.

In any of these embodiments, the dietary recommendation may be generated based on a previous ketosis score for the user.

According to some embodiments, a system for determining a level of ketosis of a user includes an acetone detector comprising an inlet for receiving a breath of a user and a sensor for sensing an amount of acetone in the breath of the user; and an electronic device comprising a display, one or more processors, memory, and one or more programs configured for execution by the one or more processors, the one or more programs including instructions for: establishing a communication connection with the acetone detector, receiving status information from the acetone detector, providing instructions on the display of the electronic device for using the acetone detector, wherein at least one instruction is displayed in response to the status information received from the acetone detector, receiving from the acetone detector one or more measurements of an amount of acetone in breath of a user, determining a ketosis score based on at least one of the one or more measurements and one or more predetermined thresholds that are associated with levels of ketosis, wherein the ketosis score is an estimate of a level of ketosis of the user, and displaying the ketosis score to the user.

In any of these embodiments, the acetone detector and the electronic device may communicate wirelessly.

In any of these embodiments, the status information received from the acetone detector may include an indication that the acetone detector is ready for measuring after an initialization period.

In any of these embodiments, the status information received from the acetone detector may include an indication that the user is blowing into the acetone detector.

In any of these embodiments, the status information received from the acetone detector may include an indication that the user has reached an end of the breath.

According to some embodiments, a method for determining a level of ketosis of a user on an electronic device includes establishing a communication connection with a acetone detector; receiving status information from the acetone detector; providing instructions on a display of the electronic device for using the acetone detector, wherein at least one instruction is displayed in response to the status information received from the acetone detector; receiving from the acetone detector one or more measurements of an amount of acetone in breath of a user; determining a ketosis score based on at least one of the one or more measurements and one or more predetermined thresholds that are associated with levels of ketosis, wherein the ketosis score is an estimate of a level of ketosis of the user; and displaying the ketosis score to the user.

In any of these embodiments, the electronic device may receive data wirelessly from the acetone detector.

In any of these embodiments, the status information received from the acetone detector may include an indication that the acetone detector is ready for measuring after an initialization period.

In any of these embodiments, the status information received from the acetone detector may include an indication that the user is blowing into the acetone detector.

In any of these embodiments, the status information received from the acetone detector may include an indication that the user has reached an end of the breath.

According to some embodiments, a system for providing a dietary recommendation to a user based on a level of ketosis of a user includes an acetone detector comprising an inlet for receiving a breath of a user and a sensor for sensing an amount of acetone in the breath of the user; and an electronic device comprising a display, one or more processors, memory, and one or more programs configured for execution by the one or more processors, the one or more programs including instructions for: receiving from the acetone detector one or more measurements of an amount of acetone in breath of a user, determining a ketosis score based on at least one of the one or more measurements and one or more predetermined thresholds that are associated with levels of ketosis, wherein the ketosis score is an estimate of a level of ketosis of the user, transmitting the ketosis score over a communication network to a server system, receiving a dietary recommendation from the server system, wherein the dietary recommendation is based on the ketosis score, displaying the dietary recommendation to the user, receiving a user selection associated with the dietary recommendation, and in response to receiving the user selection, sending an instruction to the server system to purchase at least one item associated with the recommendation.

In any of these embodiments, the acetone detector and the electronic device may communicate wirelessly.

In any of these embodiments, the at least one item may be a prepared meal.

In any of these embodiments, the at least one item may be a grocery item.

In any of these embodiments, the instruction to the server system to purchase the at least one item may be an instruction to purchase the at least one item through a third party system.

According to some embodiments, a method for providing a dietary recommendation to a user based on a level of ketosis of a user includes receiving from an acetone detector one or more measurements of an amount of acetone in breath of a user; determining a ketosis score based on at least one of the one or more measurements and one or more predetermined thresholds that are associated with levels of ketosis, wherein the ketosis score is an estimate of a level of ketosis of the user; transmitting the ketosis score over a communication network to a server system; receiving a dietary recommendation from the server system, wherein the dietary recommendation is based on the ketosis score; displaying the dietary recommendation to the user; receiving a user selection of the dietary recommendation; and in response to receiving the user selection of the dietary recommendation, sending an instruction to the server system to purchase at least one item associated with the recommendation.

In any of these embodiments, the electronic device may communicate wirelessly with the acetone detector.

In any of these embodiments, the at least one item may be a prepared meal.

In any of these embodiments, the at least one item may be a grocery item.

In any of these embodiments, the instruction to the server system to purchase the at least one item may be an instruction to purchase the at least one item through a third party system.

### BRIEF DESCRIPTION OF THE DRAWINGS

The invention will now be described, by way of example only, with reference to the accompanying drawings, in which:

FIG. 1 shows a system for monitoring and managing a ketosis level of a user, according to some embodiments;

FIG. 2 shows a block diagram of a breath analyzer for measuring the acetone in a user's breath, according to some embodiments;

FIG. 3 is a flow diagram illustrating a method 300 for measuring the amount of acetone in the breath of a user and generating acetone-based dietary recommendations, according to some embodiments;

FIGS. 4A-4E are exemplary user interfaces for guiding a user through a breath acetone measurement sequence, according to some embodiments;

FIG. 5 is an exemplary user interface for providing a dietary recommendation to a user, according to some embodiments;

FIG. 6 is a method for determining a ketosis score for a user based on a breath acetone measurement, according to some embodiments;

FIG. 7 is an exemplary user interface for providing a user with a ketosis score, according to some embodiments;

FIG. 8 is a an exemplary portion of a meal and food database for providing ketosis level-based recommendations, according to some embodiments;

FIG. 9 illustrates an exemplary user interface for ordering recommended meal and/or order ingredients for a recommended meal, according to some embodiments; and

FIG. 10 illustrates a computing device, according to some embodiments.

### DETAILED DESCRIPTION OF THE INVENTION

Described herein are systems and methods for ketosis-based diet monitoring and management. A user's breath acetone levels are measured through the use of a portable breath analyzer. Breath acetone measurements are used to provide the user with feedback regarding the user's level of ketosis as a proxy for a level of fat burning and to provide the user with dietary recommendations to achieve the user's dieting goal(s). Breath acetone level can be a better indicator of fat loss than other dieting metrics, such as weight, and providing a user with an indication of the user's level of ketosis can help a user stay motivated in their dieting and better informed regarding how the user's dietary choices affect the user's weight loss. Providing a user with dietary recommendations can help a user make dietary choices that lead to achievement of the user's weight loss goals. According to some embodiments, systems and methods can provide users with the ability to order recommended foods and/or meals, further helping users make the right dietary choices and stick with the dietary programs. Through these mechanisms, the systems and methods described herein can lead to improved, personalized dietary programs and can help motivate users to stick with their dietary programs.

In the following description of the disclosure and embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present invention include process steps and instructions described herein in the form of an algorithm. It should be noted that the process steps and instructions of the present invention could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present invention also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

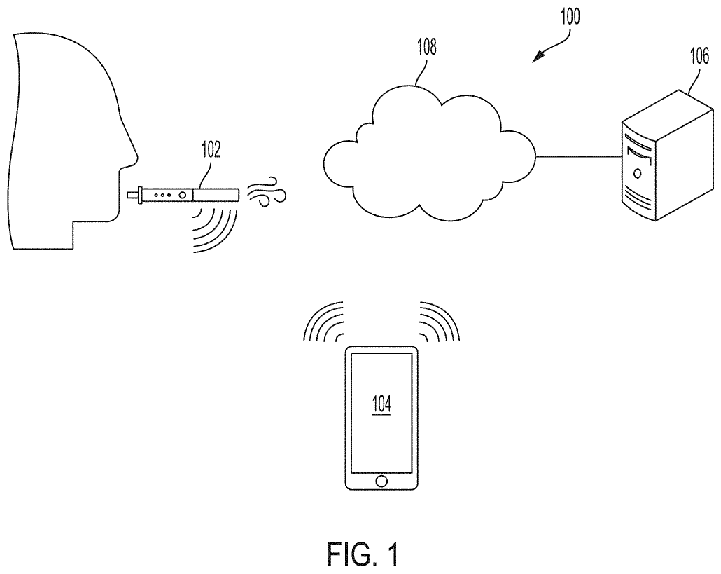

FIG. 1 illustrates a system 100 for monitoring and managing a ketosis level of a user. System 100 includes a breath analyzer 102 for measuring the amount of acetone in a user's breath and a portable electronic device 104 for monitoring and managing the user's ketosis levels using the acetone measurements from the breath analyzer 102.

The breath analyzer 102 can be a portable device that the user can take with them and use repeatedly throughout the day for measuring acetone levels at different times. The portable electronic device 104 can be a smartphone, tablet, laptop, smartwatch, or any other suitable device that may run a ketosis monitoring software application (App) for receiving acetone measurements from the breath analyzer 102 and using the measurements for assessing the user's level of ketosis.

The App can provide useful information to the user to help the user track and manage their diet. Such information can include current ketosis level, which can help a user gauge whether a desired ketosis level is being achieved. According to some embodiments, the App can provide dietary and/or lifestyle recommendations for achieving ketosis level goals. Recommendations can be generated based on the acetone measurements and based on user-specific factors such as user attributes (e.g., weight, dietary preferences, duration on program) and user goals (e.g., weight loss, weight maintenance).

In some embodiments, the portable electronic device 104 is communicatively connected to a server system 106 (e.g., a cloud service) via one or more networks 108. User information may be stored in one or more databases associated with the server system 106. The App can communicate the user's level of ketosis to the server system 106. The server system 106 can generate recommendations for the user and transmit the recommendations to the App for display to the user. The server system 106 can facilitate other functionality of the App, including social media related capabilities for interconnecting a community of App users.

To measure the amount of acetone in the user's breath, the user blows into the breath analyzer 102. As discussed in more detail below, a sensor in the breath analyzer 102 generates a signal that is based on the amount of acetone in the user's breath. The breath analyzer 102 converts the signal to one or more digital measurements that are provided to the portable electronic device 104. The breath analyzer 102 and portable electronic device 104 can communicate via a wired or wireless connection. Preferably, the breath analyzer 102 and portable electronic device 104 communicate wirelessly (e.g., via Bluetooth, ZigBee, IEEE 802.11x, etc.). Once connected to the portable electronic device 104, the breath analyzer 102 can send acetone measurements to the portable electronic device 104.

In some embodiments, the App can provide guidance to the user on using the breath analyzer 102. This guidance can be based on data received from the breath analyzer 102. For example, the breath analyzer 102 may send an indication to the portable electronic device 104 that the breath analyzer is ready for use, and the App may display a notification to the user that the user can begin blowing into the breath analyzer.

The App uses acetone measurements from the breath analyzer to determine a score associated with the level of ketosis of the user. The ketosis score can be displayed to the user on a display of the portable electronic device so that the user can assess the status of the user's diet. The App can also use the ketosis score to generate diet-based recommendations for the user. The dietary guidance can be generated based on a user-specific goal, such as a desired amount of weight loss, and user-specific attributes, such as weight, gender, dietary preferences and age. The App may provide a user with recommended meals or may suggest to the user that various foods be eaten or avoided.

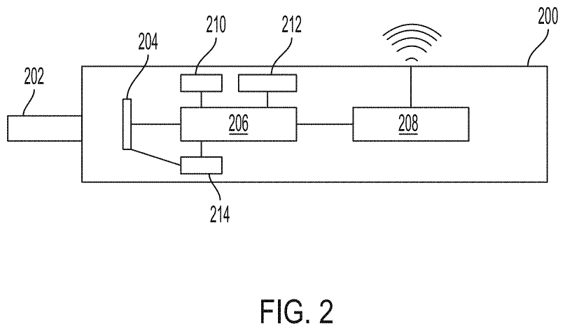

FIG. 2 illustrates an exemplary breath analyzer 200, according to one embodiment. Breath analyzer 200 includes a breath inlet 202 for receiving a user's breath, a sensor 204 for sensing an amount of acetone in the breath received through the breath inlet 202, one or more processors 206 for processing signals from the sensor 204 to generate acetone measurements, and a communication interface 208 for communicating (e.g., wirelessly) with a remote device, such as portable electronic device 104 of FIG. 1, to provide the acetone measurements.

In some embodiments, the breath analyzer 200 is a handheld device. The user can bring the device up to the user's mouth to blow into the breath inlet 202. In other embodiments, the breath analyzer is a table-top device or includes a table-top portion in addition to a separate handheld portion for blowing into. The breath analyzer 200 may be a portable device that a user can take with them, for example, to use outside of the home. A handheld, portable breath analyzer enables a user to have the analyzer with them throughout the day for taking multiple times in measurements in a day and can ensure regular use.

In use, a user exhales into the breath inlet 202. The breath flows over the sensor 204. The sensor 204 generates a signal that is proportional to the amount of acetone in the breath flowing over the sensor 204. The processor(s) 206 generate one or more measurements from the signal generated by the sensor. The communication interface 208 establishes a communication connection with a portable electronic device, such a user's smartphone, or to a personal computer or other computing device, and transmits one or more of the measurements to the external device.

The sensor 204 is configured to sense the amount of acetone in the breath of the user. Any suitable sensor or sensing system may be used. In some embodiments, the sensor 204 is a metal oxide semiconductor sensor that is specific for acetone. The sensor may be made of tungsten, tin, aluminum, silicon, silicone, carbon, oxygen, and other metals and compounds, and configured so that the surfaces of the metal oxide nanoparticles react with oxygen from the air and reducing gases such as acetone, resulting in conductivity changes that are measured. The level of conductivity change produced during a breath measurement is turned into a digital signal. This digital signal is received by a microcontroller 206, and transmitted by a communication interface 208 to the app.

Processor 206 converts the analog current signals from the sensor into digital measurements of the amount of acetone in the user's breath, using well-known analog to digital conversion techniques and hardware. The result of the digital conversion is often referred to herein as raw data. In some embodiments, the one or more processors transform the raw data into the one or more measurements, such as for filtering noise or removing a sensor offset. In some embodiments, the measurements are uncalibrated such that they include sensor-specific biases. The uncalibrated measurements are transmitted to the external device and calibration of the measurements is performed by the external device.

In some embodiments, breath analyzer specific calibration parameters are stored on a memory 212 of the analyzer and communicated to the external device for calibration of the measurements by the external device. Analyzer specific calibration parameters are parameters that have been determined for the specific analyzer, such as through laboratory or factory testing of the analyzer before delivery to a user. Analyzer specific calibration parameters can be generated, for example, by exposing the breath analyzer to a known concentration of acetone and determining the difference between the data generated by the analyzer and the known concentration. Analyzer specific calibration parameters are those that will adjust the analyzer data to the known concentrations, according to one or more predetermined conversion functions. For example, in some embodiments, measurements generated from the sensor signal may have an offset and the calibration parameter(s) provided to the external device may include the amount of the offset. The external device may subtract the offset from the acetone measurements received from the analyzer to generate calibrated measurements.

In some embodiments, the processor 206 may calibrate the measurements so that calibrated measurements are provided to the external device. Using the example above, the processor 206 may use the amount of the offset to adjust the measurements generated by the raw conversion of the sensor signals.

Calibration parameter(s) may be determined at the factory by exposing the sensor 204 to one or more levels of known concentrations of acetone and determining a difference between the raw data from the sensor and the known concentration. One or more calibration parameters may be generated based on the difference and these parameters may be stored in the memory 212 for use by the processor 206 for generating calibrated measurements or for transmission to the external device along with the raw data for calibration of the data by the external device. The calibration parameter(s) may also be stored in the server system, and used by the external device along with raw data to produce a calibrated measurement.

The communication interface 208 provides a communication connection with an external device for communicating the acetone measurements and any other relevant information to the external device. The communication interface 208 may be a wireless network interface that may be coupled to a wireless antenna for communicating acetone measurements wirelessly to the portable electronic device (or any other external device).

In some embodiments, the breath analyzer includes a display 210. The display 210 can be used to communicate information to the user. In some embodiments, the display 210 is used to display analyzer status information. The display 210 can be one or more indicator lights, such as LEDs, that indicate analyzer status. The display 210 can be or include an LCD screen for displaying numbers or text. In some embodiments, one or more lights are used to indicate analyzer status, such as a ready light for indicating that the analyzer is not ready for use, and/or a green light to indicate that the analyzer is ready. A series of multiple lights may be used to indicate progress to a state, such as progress to a ready-to-use state or progress to a measurement cycle completion state. In some embodiments, one or more indicators are used to indicate battery life, an on/off state, and/or a wireless connection state. In some embodiments, an LCD screen may be used to provide any relevant information, including states of the device (on/off, ready-for-use, low battery, etc.), and acetone measurements.

The breath analyzer 200 may include one or more additional sensors 214 for facilitating acetone measurements. In some embodiments, the breath analyzer includes a temperature sensor that can be used for managing a temperature sensitivity of the acetone sensor. In embodiments in which the signal generated by the acetone sensor varies with temperature of the sensor, the temperature sensor may be used to monitor the temperature of the breath analyzer. Temperature data can be used to indicate to the user when the analyzer is ready for use and/or can be used to adjust the acetone measurements. The monitored temperature may be used to notify a user when the analyzer is ready for use. For example, the breath analyzer may send an indication to the portable electronic device that the breath analyzer is ready for use based at least in part of the sensor reaching a predetermined temperature. In some embodiments, the breath analyzer 200 includes a heating system for controlling the temperature of the acetone sensor 204.

In some embodiments, the analyzer does not have a temperature sensor, but may still account for a temperature sensitivity by waiting for a predetermined time after start-up before indicating to the user (e.g., via the display 210 or via an indication sent to the portable electronic device) that the analyzer is ready to use. For example, it may be determined that the analyzer will warm to a satisfactory temperature in a predetermined period of time, and the display 210 may indicate that the analyzer is ready to use only after the predetermined time has elapsed or the breath analyzer may transmit a ready to use signal to the portable electronic device only after the predetermined time has elapsed.

In some embodiments, the breath analyzer includes a flow sensor for sensing whether a user is blowing into the breath inlet 202. The flow sensor may be any suitable type of sensor for sensing that a user is blowing into the analyzer. In some embodiments, the flow sensor is a pressure sensor that senses the pressure of the breath created by the user exhaling. In other embodiments, the flow sensor includes a turbine that rotates in response to fluid flow. In some embodiments, data from the flow sensor (e.g., pressure sensor) may be transmitted to the portable electronic device as an indication of the usage of the breath analyzer. In other embodiments, the breath analyzer may use the data to generate and usage status indication, which is then provided to the portable electronic device and/or displayed via display 210.

As stated above, the breath analyzer, such as breath analyzer 200, communicates with a portable electronic device, such as a smartphone, tablet, or smartwatch, executing an App. According to some embodiments, the App can assist the user in using the breath analyzer, can determine a ketosis score that reflects the level of ketosis of the user based on measurements from the breath analyzer, and can provide recommendation to the user based on the ketosis score.

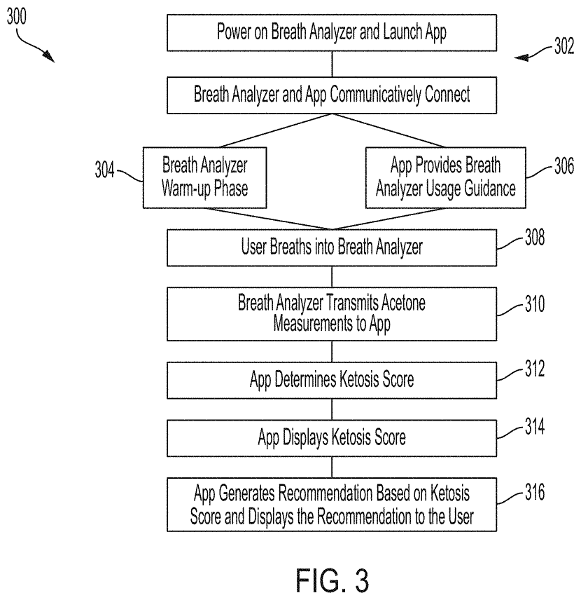

FIG. 3 is a flow diagram illustrating a method 300 for measuring the amount of acetone in the breath of a user and generating acetone-based dietary recommendations, according to one embodiment. At step 302, the App is launched on the portable electronic device, and the breath analyzer is powered on. The App and breath analyzer can automatically communicatively connect to one another, such as using Bluetooth or other wireless communication technology or a wired communication technology.

At step 304, the breath analyzer enters a warm-up phase. In the warm-up phase, the analyzer may perform various calibration and/or stabilization techniques to ensure that subsequent acetone measurements are accurate. For example, the breath analyzer may perform controlled heating of the sensor to ensure that the sensor is at or near a predefined temperature.

In some embodiments, the sensor is maintained at or near a sensing temperature continuously or semi-continuously in order to reduce the delay before the user can blow into the analyzer, which may eliminate or significantly shorten the warm-up phase. A heating system within the analyzer can monitor the temperature of the sensor and maintain the operating temperature of the sensor or maintain a lower temperature that can be quickly increased to the operating temperature when needed. In some embodiments, the analyzer can be selectively placed in such a “fast” heat-up mode, such as via a selection in the App. In some embodiments, when the analyzer is in the fast heat-up mode, the user may indicate via an input to the App that the user wants to measure their ketosis level and the App may indicate to the user to blow into the analyzer within a short period of time, which can be, for example, within less than 30 seconds, preferably within less than 20 seconds, more preferably, within less than 10 seconds, more preferably within 5 seconds, more preferably within 2 seconds, and more preferably within 1 second.

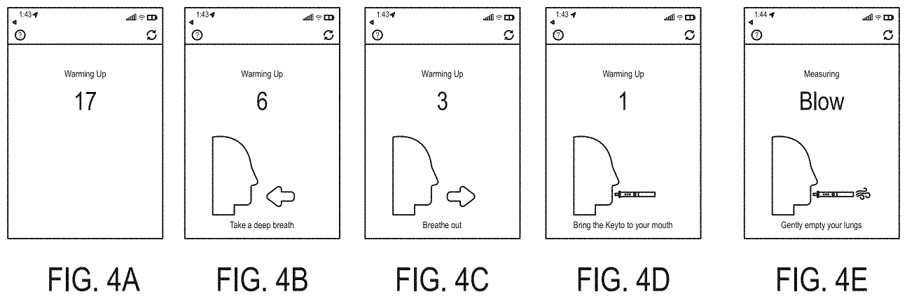

At step 306, the App may provide notifications to the user regarding the use of the breath analyzer. This step may be performed while the breath analyzer is in the warm-up phase. Exemplary notification screens are illustrated in FIGS. 4A-E. As shown in FIG. 4A, the App may inform the user that the breath analyzer is in the warm-up phase. The App may provide an indication or estimate of the time until the breath analyzer is ready to use (e.g., the number of seconds remaining as in FIG. 4A, or a completion percentage). The App may communicate with the breath analyzer to estimate the amount of time before the breath analyzer is ready for use. For example, the breath analyzer may send a notification to the App that it is beginning the warm-up period and the App may initiate a countdown timer that is associated with a predetermined warm-up phase time period. In other embodiments, the breath analyzer may send status information periodically to the App to inform the App of the progress of, for example, the warm-up phase of the breath analyzer. The App may adjust the breath analyzer status display to the user based on the updates from the breath analyzer.

In some embodiments, such as in some embodiments that include a fast heat-up mode, the App may simply provide an indication that the analyzer is ready to use. For example, upon a user input to the App indicating that the user desires to take a measurement, the App may respond to the user input by providing a message that the analyzer is ready for the user to blow into the analyzer and/or may provide a short countdown—e.g., “5, 4, 3, 2, 1, Blow”).

As shown in FIG. 4B, the App may guide the user to begin the process of blowing into the breath analyzer by instructing the user to take a breath. This guidance may be provided at a predefined time from the start of the warm-up period (or a predefined time to the end of the warm-up period) or may be provided once the warm-up is complete and the breath analyzer is ready for use. The App may then instruct the user to breathe out, as shown in FIG. 4C, and then to bring the breath analyzer to the user's mouth while continuing to blow, as shown in FIG. 4D.

At step 308, the user breaths into the breath analyzer, for example, as guided by the App in the manner described above or other suitable manner. The App may then instruct the user to continue to blow until the user is out of breath, as shown in FIG. 4E. Blowing until the end of the breath may be important in ensuring that acetone in the user's lungs reaches the sensor, since acetone is typically present in the alveoli and alveolar breath ensures that acetone is evacuated. In some embodiments, the App instructs the user to stop blowing into the breath analyzer before the end of the breath.

In some embodiments, the guidance provided to the user may be based on information received from the breath analyzer. The breath analyzer may sense one or more attributes of the user's breath that may indicate how well the user is using the breath analyzer and may provide information to the App that is associated with the user's use of the breath analyzer. For example, the breath analyzer may measure a pressure associated with the user blowing into the breath analyzer and may provide this pressure, or an indication generated based on the pressure, to the App. The App may determine that the user has stopped blowing into the App and may indicate to the user that the length of the exhale into the breath analyzer was insufficient to generate a good measurement. The App may then recommend that the user re-measure.

During the user's exhale into the breath analyzer, the sensor in the breath analyzer senses the amount of acetone in the user's breath. The breath analyzer converts the sensor signal into one or more digital measurements that are associated with the amount of acetone in the user's breath that reaches the sensor.

At step 310, the breath analyzer transmits one or more breath acetone measurements to the App. In some embodiments, the breath analyzer transmits a series of measurements that include multiple measurements generated during the delivery of the breath by the user. The breath analyzer may transmit the series as a stream during the breath delivery or once the measurement session has ended. In other embodiments, the breath analyzer transmits a single measurement. This single measurement may be a peak measurement or may be a value that is based on the peak measurement (e.g., a value that is a predefined threshold percentage of a peak measurement or higher than a predetermined percentage of acetone measurement values).

In some embodiments, the breath analyzer transmits a baseline measurement to the App, which the App may use to adjust the one or more acetone measurements received from the App. This baseline measurement may be generated prior to the user blowing into the breath analyzer and may be associated with a baseline signal from the sensor. In some embodiments, the baseline measurement is provided to the App prior to measurement of the user's breath. In other embodiments, the baseline measurement is provided along with the maximum acetone measurement.

In some embodiments, the acetone measurements provided to the App are adjusted by the breath analyzer prior to transmission using the baseline measurement. For example, the baseline measurement may be subtracted from the raw data generated from the acetone sensor signal by the one or more processors of the breath analyzer. The adjusted data may then be provided to the App. In other embodiments, the App may perform this adjustment in response to receiving the baseline measurement and one or more acetone measurements.

At step 312, the App determines a ketosis score based on the acetone measurement data received from the breath analyzer. According to some embodiments, the ketosis score is generated by converting the raw data received from the breath analyzer to a values associated with predefined measurement scale. For example, the raw data may be converted to parts-per-million (PPM) values. The score may then be determined by comparing the PPM value(s) or to values generated from the PPM values to predetermined thresholds. In some embodiments, the score is a number associated with a predefined level of acetone in the user's breath. The score may be a number from 1-3, 1-5, 1-6, 1-8, 1-10, 1-15, 1-20, 1-50, or any other suitable range of numbers. Each score may be associated with lower and upper thresholds. For example, a score of 1 may be associated with PPM values that are below 5 PPM, a score of 2 may be associated with PPM values from 5 to 10, a score of 3, may be associated with PPM values from 10-15, and so on. In some embodiments, the scores are qualitative, rather than quantitative. For example, a range of scores may be low, moderate, high, and very high.

The thresholds defining scores can be based on clinical data associated with breath acetone levels for test subjects undergoing various levels of ketosis. For example, a ketosis score of “low” or a low number may be associated with breath acetone levels for test subjects who recently consumed carbohydrates and, thus, are likely metabolizing little if any fat. A score of “very high” or a high number may be associated with breath acetone levels for test subjects that have unhealthy levels of ketones in their bloodstream, such as subjects experiencing diabetic ketoacidosis.

According to some embodiments, the App determines a score for a given breath analysis by comparing the acetone measurement or a value that is based on the acetone measurement, such as the PPM value or a value based on the PPM value, to the thresholds defining the scores. For example, a PPM value of 10 that results from a breath acetone measurement may correspond to a “moderate” score.

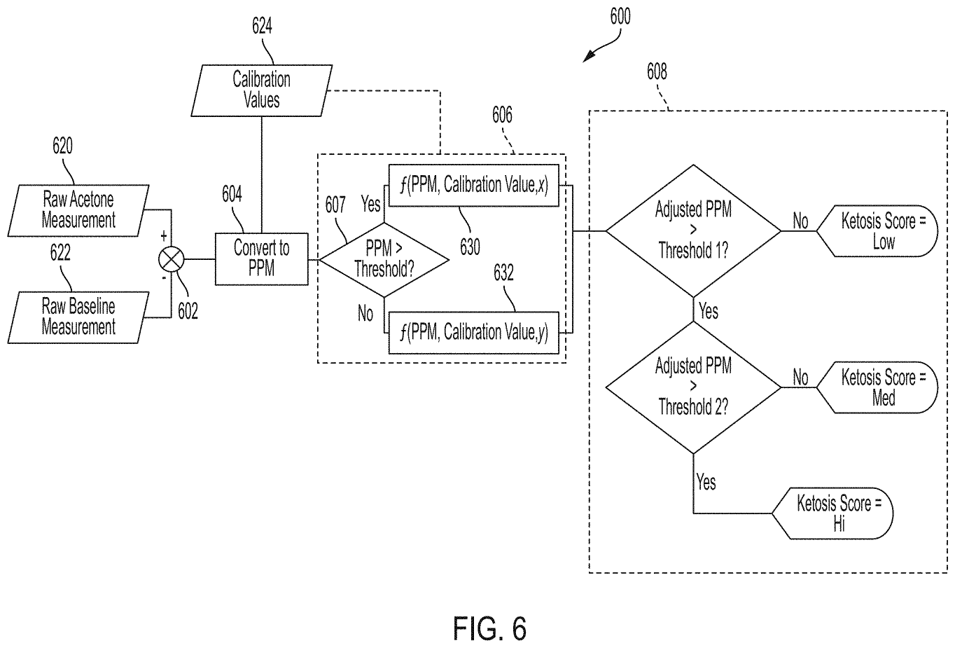

FIG. 6 illustrates a method 600 for determining a ketosis score from a breath analyzer sensor measurement, according to one embodiment. Method 600 may be performed entirely by the App on the portable electronic device. In other embodiments, one or more steps of method 600 may be performed by the breath analyzer.

At step 602, a difference between a raw breath measurement value 620 and a raw baseline measurement value 622 is computed, resulting in a corrected breath measurement value. In this embodiment, the raw breath measurement value 620 is a value resulting from an analog to digital conversion of the breath analyzer sensor signal. The raw baseline measurement value 622 can be generated during a warm-up, calibration, or idle phase of the breath analyzer before the user exhaled into the device, and represents the portion of the sensor signal that is not attributable to the presence of acetone. In some embodiments, step 602 can be performed by the breath analyzer and the resulting corrected breath measurement value may be provided to the App. In other embodiments, the raw breath measurement value and the raw baseline measurement value are provided to the App and the App calculates the corrected breath measurement value.

At step 604, the corrected breath measurement value is converted to a PPM value using one or more calibration parameters 624 from the breath analyzer. In some embodiments, the calibration parameters are received from the breath analyzer along with the raw breath measurement value. In some embodiments, the calibration parameters are stored by the App or in the server for future use so that the parameters do not need to be received during a subsequent measurement.

At step 606, the calibrated measurement (PPM) value may be adjusted in a manner that amplifies differences between values. For example, PPM values can be adjusted using a power function or other suitable adjusting function. In some embodiments, the calibrated measurement can be adjusted as a function of one or more calibration values received from the breath analyzer (e.g., calibration values determined at the factory). In some embodiments, the function used to adjust a PPM value may depend on whether the PPM value is above or below a threshold. In some embodiments, different functions may be applied depending on where the PPM value is in comparison to one or more thresholds. For example, the PPM value can be compared to a threshold at step 607 and a first function 630 may be applied to PPM values below the threshold (e.g., a threshold of 5 PPM) and a second function 632 may be applied to PPM values above the threshold. In the illustrated embodiment, PPM values above a threshold are adjusted based on one or more calibration values and an adjustment factor x in step 630 and PPM values below the threshold are adjusted based on the one or more calibration values and a different adjustment factor y in step 632.

At step 608, a ketosis score is determined by comparing the adjusted PPM value to a plurality of threshold values that define ketosis score bins. Any number of bins may be defined. For example, 100 bins or less may be defined, 50 bins or less may be defined, 25 bins or less may be defined, 10 bins or less may be defined, 8 bins or less may be defined, 6 bins or less may be defined, 5 bins or less may be defined, or 3 bins or less may be defined. 3 bins (low, medium, and high) are illustrated in FIG. 6.

Each bin may be defined by upper and lower threshold values. For example, a first bin may be associated with values of 0 to 5 adjusted PPM, a second bin may be associated with values of 5-10 adjusted PPM, a third bin may be associated with values of 10-15 adjusted PPM, and so on. The adjusted PPM value (or unadjusted value) may be compared to one or more thresholds to determine the bin that the value is associated with. The ketosis score for a given value is the ketosis score that is associated with the bin that the given value fits within.

Bins need not be the same size. Larger bins (i.e., bins encompass a greater range of converted PPM values) may be provided at the lower end and/or the upper end of converted PPM values. A bin that captures the upper end of PPM values may be sized to encompass all non-healthy levels of acetone. Similarly, a bin that captures the lower end of PPM values may be sized to encompass acetone levels that indicate little or no fat metabolism. Multiple bins may be provided between these two extremes so as to capture the range of fat burning with more granularity while still providing qualitative usefulness. For example, where an adjusted PPM value of 6 has little practical difference in terms of the effectiveness of a user's diet in initiating fat metabolism from an adjusted PPM value of 7, these values may be part of the same bin.

In some embodiments, the ketosis score is not based on any attribute of the user but, rather, is based only on the acetone measurement, such as the ketosis score generated in method 600. In other words, the ketosis score is independent of attributes of the user. In other embodiments, the ketosis score is based on additional factors beyond the acetone measurement. For example, different score thresholds may be provided for different genders, different ages, or any other relevant human attribute. So, the same score of “high” for a child and an adult may reflect different breath acetone levels.

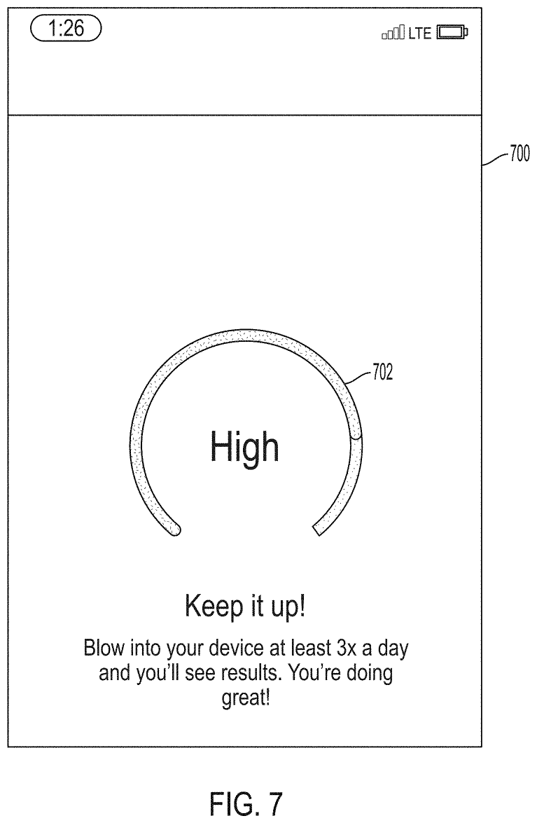

Returning to method 300 of FIG. 3, at step 314, the App may display the score associated with the acetone measurement to the user. For example, the App may display a qualitative score of “moderate” or a quantitative score of “3” to the user. This can help a user understand their body's current ketosis level. A user can learn from the ketosis score how the user's diet affects the level of ketosis and how the level of ketosis changes throughout the day and over days, weeks, and months. An exemplary ketosis score display user interface 700 is illustrated in FIG. 7 with a ketosis score 702 of “High” displayed.

At step 316, the App may use the ketosis score to generate recommendations for the user, which are then displayed to the user. Recommendations can include dietary recommendations, exercise recommendations, breath analyzer usage recommendations, and any other suitable recommendation. The App (alone or in combination with a connected server system) may generate recommendations based on attributes of the user. Examples of user attributes that can be used to generate a recommendation include gender, weight, age, dietary preferences, and weight loss goal. A weight loss goal can be an amount of weight to lose over a period of time, a percentage of weight to lose over a period of time, maintaining weight, or any suitable goal associated with weight management. The App may include one or more data-entry user interfaces for a user to provide user attributes.

According to some embodiments, the App can generate recommendations based on one or more previous ketosis scores. For example, a current ketosis score that is higher than a previous ketosis score may result in a recommendation for the user to continue with the user's current diet, whereas, a current ketosis score that is lower than a previous ketosis score may result in a recommendation for the user to decrease the amount of carbohydrates in the user's diet. The App may use the previously generated ketosis score, a ketosis score generated around the same time the previous day, a ketosis score generated on the same day the previous week, or a ketosis score from any other suitable period.

Recommendations generated by the App may include general qualitative guidance, such as a recommendation to keep up the user's current diet. This type of recommendation may be generated when a user's ketosis score reflects a level of ketosis that if maintained will likely result in the user meeting their defined goal. Another example of a qualitative recommendation is an instruction to eat fewer carbohydrates. This recommendation may be provided to a user that has a ketosis score that reflects a low level of ketosis.

Recommendations for specific meals or specific foods can also be provided to the user. For example, very low carbohydrate meals may be suggested to a user that has a trend of ketosis low scores (e.g., previous and current), whereas meals with moderate amounts of carbohydrates may be suggested to a user that has a good trend of ketosis score.

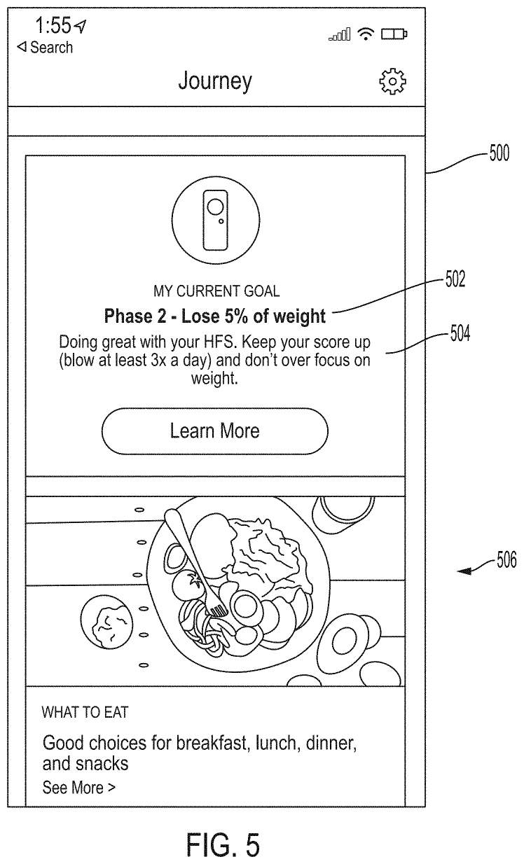

FIG. 5 illustrates an exemplary recommendation user interface 500 that may be generated by the App. The recommendation user interface 500 may include the user's current goal 502, which in this example is “Phase 2—Lose 5% of weight.” The user interface 500 includes a recommendation 504 that provides the guidance that the user is doing great, should strive to keep the score up, should use the breath analyzer three times a day, and should not focus too much on their weight (e.g., since weight changes over short periods of time are often due more to water amount changes than fat amount changes).

The user interface 500 can also include a diet recommendation section 506. The user may select this section to find specific meal or food recommendations that can help the user achieve the user's goal. For example, a user that has a high ketosis score and has a goal to lose weight may be provided with recommendations for specific meals and/or foods that are lower in carbohydrates and higher in fat than a user that has a moderate ketosis score and a similar goal.

According to some embodiments, recommendations are generated by the App, by a server system communicatively coupled to the App, such as server system 106 of system 100, or by a combination of the App and a server system. In some embodiments, the App may send one or more ketosis scores for a user to the server system and the server system may respond with one or more recommendations generated based on the one or more ketosis scores. Recommendations sent to the App by the server system may include a meal or food item identifier and may also include additional information, such as an image of the meal or food, a recipe for a meal, a meal or food ketosis rating, or any other suitable information.

Meal and food recommendations can be generated based on any suitable number of factors and combinations of factors. Factors can include current ketosis score, one or more historical ketosis scores, user attributes such as gender and weight, weight loss goals, dietary preferences, and food ratings.

Factors can also include food consumption trends, such as a user's meal or food consumption history. A user may provide their historical food consumption data via the App and the information may be used to determine what foods or meals to recommend in the future. Foods that the user appears to like most, as indicated for example by the frequency of consumption, may be recommended more frequently than other foods. A meal that a user consumed the day before may not be recommended again the next day so that the user stays interested in recommended meals.

Factors can also include user-generated ratings associated with foods or meals. For example, a user may input into the App information regarding whether the user likes certain foods or not, such as through a “Like” user interface object associated with a displayed meal or food. After trying a meal that the App previously recommended, a user may indicate through the App user interface that the user consumed the meal and may provide a rating regarding whether the user liked the meal. This data may be provided to the server system for use in future recommendations. Recommendations can also be generated based on data from other users. Meals that others like may be recommended more often than meals that others dislike. Recipes may be uploaded by users and provided as recommendations to other users. According to some embodiments, machine learning may be used to generate recommendations for users.

Food and/or meal recommendations to the user based on the user's ketosis score and, in some embodiments, other factors, such as weight loss goals, user attributes, previous scores, and any other suitable information. According to some embodiments, food and meal recommendations may be generated based on ratings assigned to foods. A rating for a food may be associated with the suitability of the food to a fat burning diet. For example, foods with low carbohydrate content may be given higher ratings than foods with higher carbohydrate content. Based on such ratings, recommendations can be generated based on a user's level of ketosis and the user's goals.

In some embodiments, the App transmits a user's ketosis score to the server and the server generates dietary recommendations based on queries to a database of rated foods. The server may transmit a recommended meal or food item to the App for display to the user.

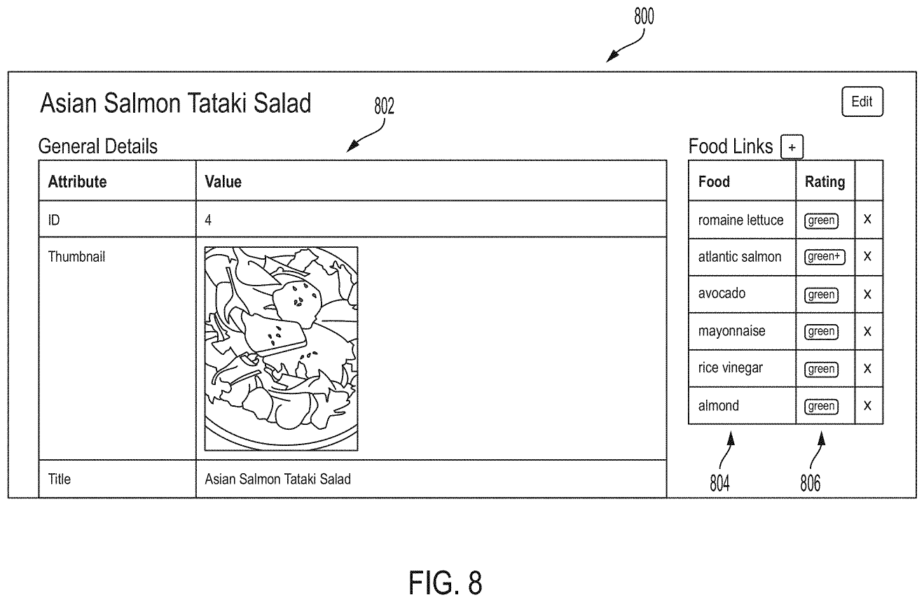

An exemplary portion of a meal and food database 800 for providing ketosis level based recommendations is provided in FIG. 8. Database 800 includes a meal 802 and a plurality of ingredients 804 that are used to make the meal 802. Each ingredient 804 includes a ketosis diet rating 806 that is associated with the ingredient's suitability for a ketosis-based diet. For example, foods that are low in carbohydrates may be rated higher than foods that are high in carbohydrates. Foods that are higher in calories that come from sources other than carbohydrates may be rated higher than foods that are lower in calories. In the example shown in FIG. 8, salmon, which is high in calories and very low in carbohydrates is rated higher than romaine lettuce, which is also very low in carbohydrates but also low in calories.

Any suitable ratings scale may be used. For example, foods may be assigned ratings from a set of less than 50 ratings, less than 30 ratings, less than 20 ratings, less than 10 ratings, or less than 5 ratings. A set of ratings may be at least 3 ratings, at least 5 ratings, at least 10 ratings, or at least 20 ratings. Ratings may be assigned based on nutritional factors, such as caloric content, carbohydrate content, protein content, and fat content. Ratings may also be assigned based on consumer tastes. For example, foods that are more likely to be enjoyed by average eaters may get a ratings bump relative to foods that are less likely to be enjoyed by average eaters. Ratings may also be based on food cost and/or availability. Any other suitable factor may be used to generate food ratings.

According to some embodiments, the App may facilitate a user's access to a recommended food or meal. For example, the App may recommend one or more meals to a user and may provide an option to order one or more of the meals, such as through a third party meal delivery service. In response to a user selecting for delivery of a recommended meal, the App may transmit the selection to the server system. The server system may contact a third party meal delivery service and place an order for the meal using the service. A database associated with the server system may store user financial information and address and may provide this information to the meal delivery service.

The App may also or alternatively facilitate a user's access to recommended meals or foods by providing the user with the option to purchase food items from a grocery store, e-commerce website, or virtual store on the App itself. For example, a recommended meal may include a recipe and the user may be able to add the recipe ingredients to a grocery store virtual shopping cart by selecting the appropriate user interface object. Upon receiving the user's selection, the App may communicate the user's selection to the server system, which may communicate with a third party grocery delivery service. A user's virtual shopping cart may be updated to include the items needed to make the recommended meal. In some embodiments, the App may provide the user with the option to add individual items of a recommended meal to a virtual shopping cart.

Through the above functionality, the App may provide the user with the ability to easily obtain a recommended meal via a simple selection in the App. This functionality may increase the likelihood that user's follow the recommendations provided to the user.

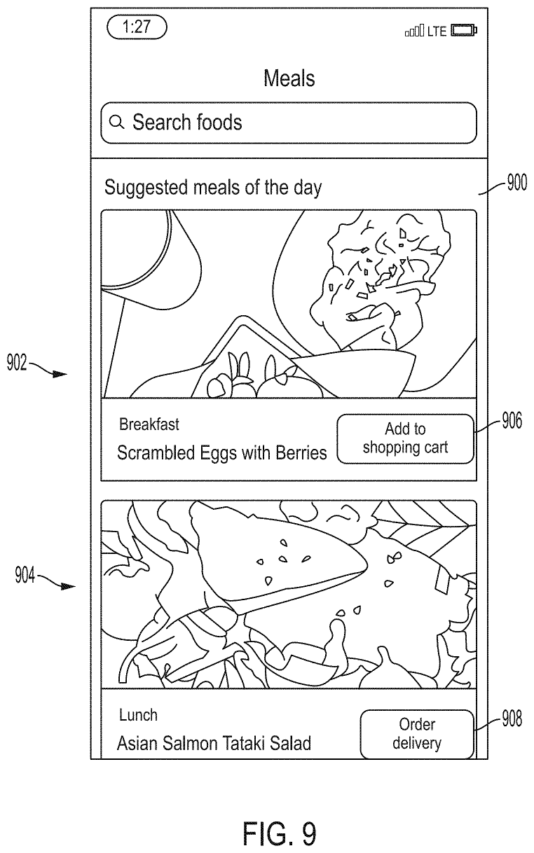

FIG. 9 illustrates an exemplary App user interface 900 for providing a user the ability to order a meal and/or order ingredients for a meal. User interface 900 includes a breakfast recommendation 902 and a lunch recommendation 904. The breakfast recommendation includes an “Add to shopping cart” selector 906. The lunch recommendation includes an “Order delivery” selector 908. Selection of selector 906 may add the ingredients for the recommended meal to a user's virtual shopping cart. The virtual shopping cart can be a shopping cart managed by the system or can be a third-party shopping cart. Selection of selector 908 may order the recommended meal, for example, via a third party meal service.

In some embodiments, selection of the selector 906 and/or 908 directly results in the items being purchased for delivery or pick-up (i.e., one-click purchase). In other embodiments, selection of the selector 906 and/or 908 may lead to one or more additional user interfaces for selecting items for addition to a virtual shopping cart and/or purchase of items in a virtual shopping cart or from a meal preparation and/or delivery service.

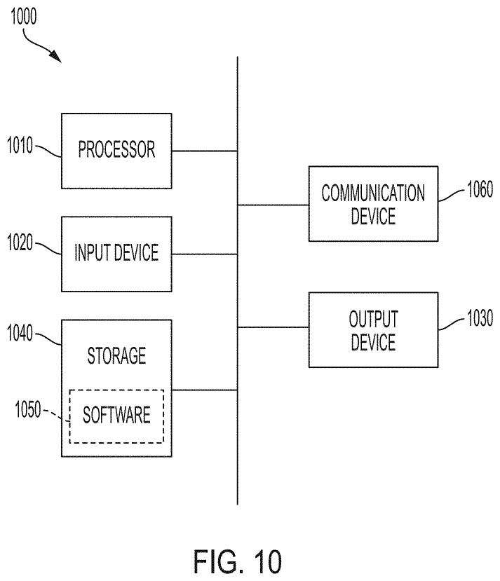

FIG. 10 illustrates an example of a computing device in accordance with one embodiment. Computing device 1000 can be a component of a system for monitoring and managing a ketosis based diet of a user, such as system 100 of FIG. 1. In some embodiments, computing device 1000 is configured to perform a method for monitoring and managing a ketosis based diet of a user, such as method 300 of FIG. 3.

Computing device 1000 can be a host computer connected to a network. Computing device 1000 can be a client computer or a server. As shown in FIG. 10, computing device 1000 can be any suitable type of microprocessor-based device, such as a personal computer, workstation, server, or handheld computing device, such as a smartphone, a tablet, or a smartwatch. The computer can include, for example, one or more of processor 1010, input device 1020, output device 1030, storage 1040, and communication device 1060. Input device 1020 and output device 1030 can generally correspond to those described above and can either be connectable or integrated with the computer.

Input device 1020 can be any suitable device that provides input, such as a touch screen or monitor, keyboard, mouse, or voice-recognition device. Output device 1030 can be any suitable device that provides output, such as a touch screen, monitor, printer, disk drive, or speaker.

Storage 1040 can be any suitable device that provides storage, such as an electrical, magnetic, or optical memory, including a RAM, cache, hard drive, CD-ROM drive, tape drive, or removable storage disk. Communication device 1060 can include any suitable device capable of transmitting and receiving signals over a network, such as a network interface chip or card. The components of the computer can be connected in any suitable manner, such as via a physical bus or wirelessly. Storage 1040 can be a non-transitory computer readable storage medium comprising one or more programs, which, when executed by one or more processors, such as processor 1010, cause the one or more processors to perform methods described herein, such as method 300 of FIG. 3.

Software 1050, which can be stored in storage 1040 and executed by processor 1010, can include, for example, the programming that embodies the functionality of the present disclosure (e.g., as embodied in the systems, computers, servers, and/or devices as described above). In some embodiments, software 1050 can include a combination of servers such as application servers and database servers.

Software 1050 can also be stored and/or transported within any computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1040, that can contain or store programming for use by or in connection with an instruction execution system, apparatus, or device.

Software 1050 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Computing device 1000 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing device 1000 can implement any operating system suitable for operating on the network. Software 1050 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The methods and systems described above can help a user lose weight by guiding a user through a ketosis based diet program. The user can be provided with metrics of the user's level of ketosis, helping a user stay motivated and informed. The user can be provided with meal recommendations and options for obtaining meals, making adherence to the ketosis based diet easier for the user and increasing the chances that the user will achieve the user's diet loss goals.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A system for determining a level of ketosis of a user, the system comprising:
an acetone detector comprising an inlet for receiving a breath of a user, a sensor for sensing an amount of acetone in the breath of the user, a memory storing one or more calibration values for calibrating sensor data, wherein the one or more calibration values are specific to the acetone detector; and
an electronic device comprising a display, one or more processors, memory, and one or more programs configured for execution by the one or more processors, the one or more programs including instructions for:
receiving from the acetone detector one or more measurements of an amount of acetone in breath of a user,
receiving from the acetone detector the one or more calibration values,
determining a ketosis score based on at least one of the one or more measurements, the one or more calibration values, and one or more predetermined thresholds that are associated with levels of ketosis, wherein the ketosis score is an estimate of a level of ketosis of the user, and
displaying the ketosis score to the user.

2. The system of claim 1, wherein the acetone detector and the electronic device communicate wirelessly.

3. The system of claim 1, wherein the ketosis score is determined by calibrating the one or more measurements using the one or more calibration values to generate one or more calibrated measurements, transforming the one or more calibrated measurements using a transform to generate one or more transformed measurements, and comparing the one or more transformed measurements the one or more thresholds.

4. The system of claim 1, wherein the ketosis score is determined based on one or more thresholds associated with levels of ketosis.

5. The system of claim 4, wherein the one or more threshold comprises no more than 10 thresholds.

6. The system of claim 1, wherein the one or more programs include instructions for transmitting the ketosis score to a remote server system that comprises at least one of dietary and lifestyle information and receiving at least one of a dietary and a lifestyle recommendation generated for the user based on the ketosis score.

7. The system of claim 6, wherein the dietary recommendation is generated based on one or more attributes of the user.

8. The system of claim 7, wherein the one or more attributes of the user comprises one or more of a weight loss goal, age, gender, dietary preferences, and weight.

9. The system of claim 6, wherein the at least one of a dietary and a lifestyle recommendation is generated based on a previous ketosis score for the user.

10. A method for determining a level of ketosis of a user on an electronic device, the method comprising:
receiving from an acetone detector one or more measurements of an amount of acetone in breath of a user;
receiving from the acetone detector one or more calibration values for calibrating acetone detector data, wherein the one or more calibration values are specific to the acetone detector;
determining a ketosis score based on at least one of the one or more measurements, the one or more calibration values, and one or more predetermined thresholds that are associated with levels of ketosis, wherein the ketosis score is an estimate of a level of ketosis of the user; and
displaying the ketosis score to the user.

11. The method of claim 10, wherein the electronic device receives data wirelessly from the acetone detector.

12. The method of claim 10, wherein the ketosis score is determined by calibrating the one or more measurements using the one or more calibration values to generate one or more calibrated measurements, transforming the one or more calibrated measurements using a transform to generate one or more transformed measurements, and comparing the one or more transformed measurements to the one or more thresholds.

13. The method of claim 10, wherein the ketosis score is determined based on one or more thresholds associated with levels of ketosis.

14. The method of claim 13, comprising no more than 10 thresholds.

15. The method of claim 10, comprising transmitting the ketosis score to a remote server system that comprises at least one of dietary information and lifestyle information and receiving at least one of a dietary and a lifestyle recommendation generated for the user based on the ketosis score.

16. The method of claim 15, wherein the at least one of a dietary and a lifestyle recommendation is generated based on one or more attributes of the user.

17. The method of claim 16, wherein the one or more attributes of the user comprises one or more of a weight loss goal, age, gender, dietary preference, and weight.

18. The method of claim 15, wherein the dietary recommendation is generated based on a previous ketosis score for the user.

19. A system for determining a level of ketosis of a user, the system comprising:
an acetone detector comprising an inlet for receiving a breath of a user and a sensor for sensing an amount of acetone in the breath of the user; and
an electronic device comprising a display, one or more processors, memory, and one or more programs configured for execution by the one or more processors, the one or more programs including instructions for:
establishing a communication connection with the acetone detector,
receiving status information from the acetone detector,
providing instructions on the display of the electronic device for using the acetone detector, wherein at least one instruction is displayed in response to the status information received from the acetone detector,
receiving from the acetone detector one or more measurements of an amount of acetone in breath of a user,
determining a ketosis score based on at least one of the one or more measurements and one or more predetermined thresholds that are associated with levels of ketosis, wherein the ketosis score is an estimate of a level of ketosis of the user, and
displaying the ketosis score to the user.

20. The system of claim 19, wherein the acetone detector and the electronic device communicate wirelessly.

21. The system of claim 19, wherein the status information received from the acetone detector comprises an indication that the acetone detector is ready for measuring after an initialization period.

22. The system of claim 19, wherein the status information received from the acetone detector comprises an indication that the user is blowing into the acetone detector.

23. The system of claim 19, wherein the status information received from the acetone detector comprises an indication that the user has reached an end of the breath.

24. A method for determining a level of ketosis of a user on an electronic device, the method comprising:
establishing a communication connection with a acetone detector;
receiving status information from the acetone detector;
providing instructions on a display of the electronic device for using the acetone detector, wherein at least one instruction is displayed in response to the status information received from the acetone detector;
receiving from the acetone detector one or more measurements of an amount of acetone in breath of a user;
determining a ketosis score based on at least one of the one or more measurements and one or more predetermined thresholds that are associated with levels of ketosis, wherein the ketosis score is an estimate of a level of ketosis of the user; and
displaying the ketosis score to the user.

25. The method of claim 24, wherein the electronic device receives data wirelessly from the acetone detector.

26. The method of claim 24, wherein the status information received from the acetone detector comprises an indication that the acetone detector is ready for measuring after an initialization period.

27. The method of claim 24, wherein the status information received from the acetone detector comprises an indication that the user is blowing into the acetone detector.

28. The method of claim 24, wherein the status information received from the acetone detector comprises an indication that the user has reached an end of the breath.

29. A system for providing a dietary recommendation to a user based on a level of ketosis of a user, the system comprising:
an acetone detector comprising an inlet for receiving a breath of a user and a sensor for sensing an amount of acetone in the breath of the user; and
an electronic device comprising a display, one or more processors, memory, and one or more programs configured for execution by the one or more processors, the one or more programs including instructions for:
receiving from the acetone detector one or more measurements of an amount of acetone in breath of a user,
determining a ketosis score based on at least one of the one or more measurements and one or more predetermined thresholds that are associated with levels of ketosis, wherein the ketosis score is an estimate of a level of ketosis of the user,
transmitting the ketosis score over a communication network to a server system,
receiving at least one of a dietary and a lifestyle recommendation from the server system, wherein the at least one of dietary recommendation is based on the ketosis score,
displaying the at least one of a dietary and a lifestyle recommendation to the user,
receiving a user selection associated with the at least one of a dietary and a lifestyle recommendation, and
in response to receiving the user selection, sending an instruction to the server system to purchase at least one item associated with the recommendation.

30. The system of claim 29, wherein the acetone detector and the electronic device communicate wirelessly.

31. The system of claim 29, wherein the at least one item is a prepared meal.

32. The system of claim 29, wherein the at least one item is a grocery item.

33. The system of claim 29, wherein the instruction to the server system to purchase the at least one item is an instruction to purchase the at least one item through a third party system.

34. A method for providing a dietary recommendation to a user based on a level of ketosis of a user, the method comprising:
receiving from an acetone detector one or more measurements of an amount of acetone in breath of a user;
determining a ketosis score based on at least one of the one or more measurements and one or more predetermined thresholds that are associated with levels of ketosis, wherein the ketosis score is an estimate of a level of ketosis of the user;
transmitting the ketosis score over a communication network to a server system;
receiving a dietary recommendation from the server system, wherein the dietary recommendation is based on the ketosis score;
displaying the dietary recommendation to the user;
receiving a user selection of the dietary recommendation; and
in response to receiving the user selection of the dietary recommendation, sending an instruction to the server system to purchase at least one item associated with the recommendation.

35. The method of claim 34, wherein the electronic device communicates wirelessly with the acetone detector.

36. The method of claim 34, wherein the at least one item is a prepared meal.

37. The method of claim 34, wherein the at least one item is a grocery item.

38. The method of claim 34, wherein the instruction to the server system to purchase the at least one item is an instruction to purchase the at least one item through a third party system.
